(19) United States
(12) Patent Application Publication
(10) Pub. No.: US 2012/0239025 A1
(43) Pub. Date: Sep. 20, 2012

(54) Isolated Current Sensor

(75) Inventor: Robert B. Smith, Loveland, CO (US)

(73) Assignee: TYCO Healthcare Group LP, Boulder, CO (US)

(21) Appl. No.: 13/050,770

(22) Filed: Mar. 17, 2011

(51) Int. Cl. A61B 18/00

(52) U.S. Cl. 606/34

(57) ABSTRACT

An electrosurgical generator is disclosed. The generator includes an output stage configured to generate a frequency electrosurgical waveform. A bridge rectifier is coupled to the output stage and configured to pass-through the radio frequency electrosurgical waveform and to transform at least a portion of the radio frequency electrosurgical waveform into direct current. The generator also includes an isolated current sensor configured to measure amplitude of the direct current.

### BACKGROUND

1. Technical Field

The present disclosure relates to electrosurgical apparatuses, systems and methods. More particularly, the present disclosure is directed to an electrosurgical apparatus having an isolated current sensor.

2. Background of Related Art

Energy-based tissue treatment is well known in the art. Various types of energy (e.g., electrical, ultrasonic, microwave, cryogenic, heat, laser, etc.) are applied to tissue to achieve a desired result. Electrosurgery involves application of high radio frequency electrical current, microwave energy or resistive heating to a surgical site to cut, ablate, coagulate or seal tissue.

In bipolar electrosurgery, one of the electrodes of the hand-held instrument functions as the active electrode and the other as the return electrode. The return electrode is placed in close proximity to the active electrode such that an electrical circuit is formed between the two electrodes (e.g., electrosurgical forceps). In this manner, the applied electrical current is limited to the body tissue positioned between the electrodes.

Bipolar electrosurgical techniques and instruments can be used to coagulate blood vessels or tissue, e.g., soft tissue structures, such as lung, brain and intestine. A surgeon can either cauterize, coagulate/desiccate and/or simply reduce or slow bleeding, by controlling the intensity, frequency and duration of the electrosurgical energy applied between the electrodes and through the tissue. In order to achieve one of these desired surgical effects without causing unwanted charring of tissue at the surgical site or causing collateral damage to adjacent tissue, e.g., thermal spread, it is necessary to control the output from the electrosurgical generator, e.g., power, waveform, voltage, current, pulse rate, etc.

In monopolar electrosurgery, the active electrode is typically a part of the surgical instrument held by the surgeon that is applied to the tissue to be treated. A patient return electrode is placed remotely from the active electrode to carry the current back to the generator and safely disperse current applied by the active electrode. The return electrodes usually have a large patient contact surface area to minimize heating at that site. Heating is caused by high current densities which directly depend on the surface area. A larger surface contact area results in lower localized heat intensity. Return electrodes are typically sized based on assumptions of the maximum current utilized during a particular surgical procedure and the duty cycle (i.e., the percentage of time the generator is on).

### SUMMARY

According to one embodiment of the present disclosure, an electrosurgical generator is disclosed. The generator includes an output stage configured to generate a frequency electrosurgical waveform. A bridge rectifier is coupled to the output stage and configured to pass-through the radio frequency electrosurgical waveform and to transform at least a portion of the radio frequency electrosurgical waveform into direct current. The generator also includes an isolated current sensor configured to measure amplitude of the direct current.

According to another embodiment of the present disclosure, an electrosurgical system is disclosed. The system includes: one or more pairs of electrodes coupled to a load; an output stage coupled to the pair of electrodes, the output stage configured to generate at least one radio frequency electrosurgical waveform; a bridge rectifier coupled to the output stage and configured to pass-through the at least one radio frequency electrosurgical waveform and to transform at least a portion of the at least one radio frequency electrosurgical waveform into direct current; and an isolated current sensor configured to measure amplitude of the direct current.

A method for electrosurgery is also contemplated by the present disclosure. The method includes: supplying at least one radio frequency electrosurgical waveform from an output stage to one or more pairs of electrodes; passing the at least one radio frequency electrosurgical waveform to the pair of electrodes through a bridge rectifier; transforming at least a portion of the at least one radio frequency electrosurgical waveform into direct current; and measuring current of the direct current.

### BRIEF DESCRIPTION OF THE DRAWINGS

Various embodiments of the present disclosure are described herein with reference to the drawings wherein:

FIG. 1 is a schematic block diagram of an electrosurgical system in accordance with the present disclosure;

FIG. 2 is a schematic diagram of the electrosurgical generator of FIG. 1 in accordance with the present disclosure;

FIG. 3 is a schematic diagram of an isolated current in accordance with the present disclosure; and

FIG. 4 is a flow chart of a method in accordance with the present disclosure.

### DETAILED DESCRIPTION

Particular embodiments of the present disclosure are described hereinbelow with reference to the accompanying drawings. In the following description, well-known functions or constructions are not described in detail to avoid obscuring the present disclosure in unnecessary detail.

A generator according to the present disclosure can perform monopolar and/or bipolar electrosurgical procedures, including vessel sealing procedures. The generator may include a plurality of outputs for interfacing with various electrosurgical instruments (e.g., a monopolar instrument, return electrode, bipolar electrosurgical forceps, footswitch, etc.). Further, the generator includes electronic circuitry configured to generate radio frequency energy specifically suited for various electrosurgical modes (e.g., cutting, blending, division, etc.) and procedures (e.g., monopolar, bipolar, vessel sealing). In embodiments, the generator may be embedded, integrated or otherwise coupled to the electrosurgical instruments providing for an all-in-one electrosurgical apparatus.

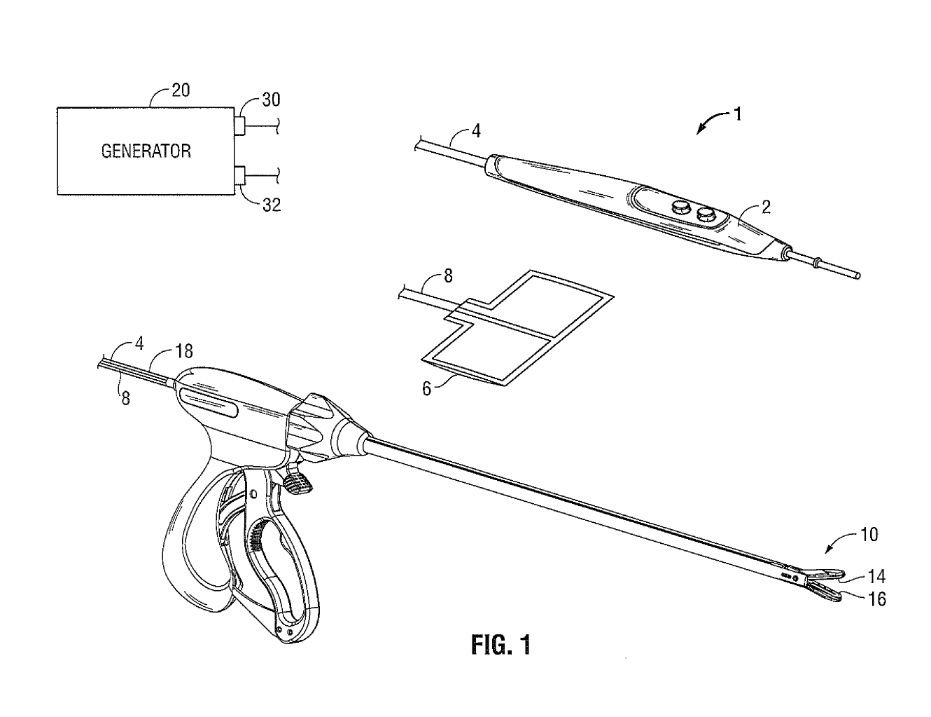

FIG. 1 is a schematic illustration of a bipolar and monopolar electrosurgical system 1 according to the present disclosure. The system 1 may include one or more monopolar electrosurgical instruments 2 having one or more electrodes (e.g., electrosurgical cutting probe, ablation electrode(s), etc.) for treating tissue of a patient. Electrosurgical energy is supplied to the instrument 2 by a generator 20 via a supply line 4 that is connected to an active terminal 30 (FIG. 3) of the generator 20, allowing the instrument 2 to coagulate, ablate and/or otherwise treat tissue. The energy is returned to the generator 20 through a return electrode 6 via a return line 8 at a return terminal 32 (FIG. 3) of the generator 20. The system 1 may include a plurality of return electrodes 6 that are disposed on a patient to minimize the chances of tissue damage by maximizing the overall contact area with the patient. In addition, the generator 20 and the return electrode 6 may be configured for monitoring so-called “tissue-to-patient” contact to insure that sufficient contact exists therebetween to further minimize chances of tissue damage.

The system 1 may also include a bipolar electrosurgical forceps 10 having one or more electrodes for treating tissue of a patient. The electrosurgical forceps 10 includes opposing jaw members having one or more active electrodes 14 and a return electrode 16 disposed therein. The active electrode 14 and the return electrode 16 are connected to the generator 20 through cable 18 that includes the supply and return lines 4, 8 coupled to the active and return terminals 30, 32, respectively. The electrosurgical forceps 10 is coupled to the generator 20 at a connector having connections to the active and return terminals 30 and 32 (e.g., pins) via a plug disposed at the end of the cable 18, wherein the plug includes contacts from the supply and return lines 4, 8.

In embodiments, the generator 20 may be configured as a portable generator and may be housed detachably or otherwise within the electrosurgical instrument 2 or the electrosurgical forceps 10. The generator 20 may be any suitable type (e.g., electrosurgical, microwave, etc.) and may include a plurality of suitable input controls (e.g., buttons, activators, switches, touch screen, etc.) for controlling the generator 20. The controls may be disposed on the generator 20 or on the electrosurgical instrument 2 or the electrosurgical forceps 10.

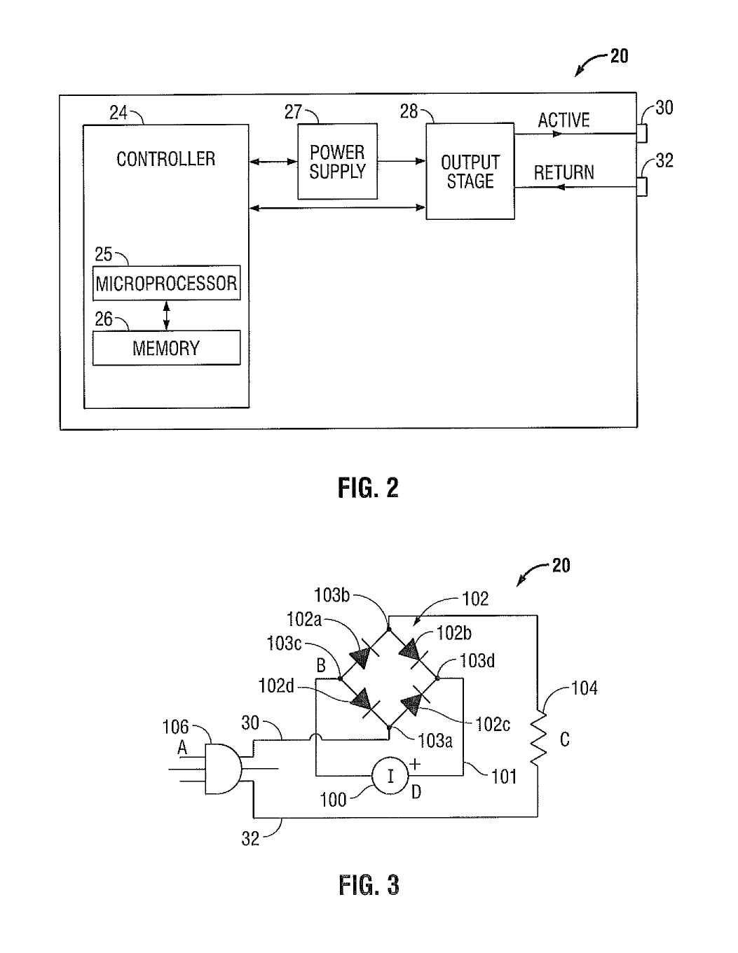

FIG. 2 shows a schematic block diagram of the generator 20 configured to output electrosurgical energy. In another embodiment, the generator 20 may be configured to output other types of energy such as, microwave, laser, etc. to power various other tissue treatment devices, such as microwave antennas, ultrasonic forceps, lasers, resistive heating electrodes, etc. The generator 20 includes a controller 24, a power supply 27, and an output stage 28. The power supply 27 may be a direct current high voltage power supply and is connected to an AC source (e.g., electrical wall outlet) and provides high voltage DC power to an output stage 28, which then converts high voltage DC power into treatment energy (e.g., ultrasonic, electrosurgical or microwave) and delivers the energy to the active terminal 30. The energy is returned thereto via the return terminal 32. The output stage 28 is configured to operate in a plurality of modes, during which the generator 20 outputs corresponding waveforms having specific duty cycles, peak voltages, crest factors, etc. In another embodiment, the generator 20 may be based on other types of suitable power supply topologies.

The controller 24 includes a microprocessor 25 operably connected to a memory 26, which may be volatile type memory (e.g., RAM) and/or non-volatile type memory (e.g., flash media, disk media, etc.). The microprocessor 25 includes an output port that is operably connected to the power supply 27 and/or output stage 28 allowing the microprocessor 25 to control the output of the generator 20 according to either open and/or closed control loop schemes. Those skilled in the art will appreciate that the microprocessor 25 may be substituted by any logic processor (e.g., control circuit) adapted to perform the calculations discussed herein.

A closed loop control scheme is a feedback control loop, in which a plurality of sensors measure a variety of tissue and energy properties (e.g., tissue impedance, tissue temperature, output power, current and/or voltage, etc.), and provide feedback to the controller 24. Such sensors are within the purview of those skilled in the art. The controller 24 then signals the power supply 27 and/or output stage 28, which then adjusts the DC and/or power supply, respectively. The controller 24 also receives input signals from the input controls of the generator 20, the instrument 2 or forceps 10. The controller 24 utilizes the input signals to adjust power outputted by the generator 20 and/or performs other control functions thereon.

Systems and methods for sensing alternating current may include very low resistive elements, such as shunt resistors, capacitors, current transformers and the like. Some of the current sensor designs are limited by safety requirements associated with electrosurgical procedures. In particular, in electrosurgical applications, voltage isolation barrier are desired or required to protect sensing circuitry from large voltages or to reduces coupled noise and interference. This prevents the use of shunt resistors and capacitors as detailed above for sensing purposes without the use of additional isolation circuit components, such as optical couplers and the like, to bridge the isolation barrier.

In one embodiment, current transformers or other sensors may be used. Current transformers may be of various sizes and varying efficiencies, particularly at high frequencies However, current transformers do not lend themselves well to miniaturization. Accordingly, a Hall Effect device or optical couplers that have a very small footprint may be used. However, such devices suffer from a low frequency response. For AC current signals above about 100 kHz, Hall effect devices are not particularly compatible, especially in higher current applications when used to sense current directly. Due to these limitations for frequencies from about 100 kHz to about 2 MHz, in embodiments from about 200 kHz to about 1 MHz, neither the current sense transformers nor the Hall effect sensors are useful when miniaturization is required.

The present disclosure overcomes the issues involved with miniaturized current sensors and provides for an isolated current sensor that is miniaturized and operates at frequencies from about 100 kHz to about 2 MHz, in embodiments from about 200 kHz to about 1 MHz. As shown in FIG. 3, the generator 20 includes an isolated current sensor 100 that is coupled to a bridge rectifier 102 that is disposed between a load 104 and a power source 106. The power source 106 represents the energy output of the output stage 28 through the active and return terminals 30 and 32. The load 104 may be any resistive load into which the energy of the power source 106 is transmitted to, such as tissue being grasped by the electrosurgical forceps 10.

The bridge rectifier 102 is shown as a frill-wave diode bridge of four diodes 102a, 102b, 102c, 102d including first and second input terminals 103a and 103b, with the first input terminal 103a coupled to the power source 106 and the second input terminal 103b coupled to the load 104 through the active terminal 30. The bridge rectifier 102 also includes first and second output terminals 103c and 103d coupled to the isolated current sensor 100 through a coupling member 101, which may be any conductive element interconnecting the first and second output terminals 103c and 103d. In embodiments, the diodes 102a, 102b, 102c, 102d may be any suitable circuit element or configuration, such as an ideal diode, configured to conduct current substantially in one direction.

During operation, the bridge rectifier 102 provides a DC current to the isolated current sensor 100 without altering the AC characteristics of the AC (e.g., RF) energy transmitted from the power source 106 to the load 104. In particular, the bridge rectifier 102 allows the AC current to pass therethrough and to convert a portion of the AC current from the power source 106 into DC current that is then sensed by the isolated current sensor 100 while the load 104 is still fed by the AC current.

Since AC current operates in a push-pull manner, during the push or positive cycle, the AC current flows from the active terminal 30 to the return terminal 32, namely, the AC current flows through the diode 102e to the isolated current sensor 100 and then through the diode 102a to the load 104 and the return terminal 32. During the pull or negative cycle, the AC current flows from the return terminal 32 to the active terminal 30, namely, the AC current flows through the diode 102b to the isolated current sensor 100 and then through the diode 102d back to the power source 106.

The pairs of diodes 102a and 102c and 102b and 102d transform the AC current into DC current, which is then measured by the isolated current sensor 100. In embodiments, the isolated current sensor 100 may be a Hall Effect sensor or any other lower frequency sensing device that does not alter the AC characteristics of the power source 106 and the load 104. The isolated current sensor 100 measures the variations in the magnetic field caused by the changes in the current transmitted through the coupling member 101. In embodiments, the isolated current sensor 100 is disposed in proximity to the coupling member 101 based on the sensitivity of the Hall Effect sensor, obviating the need for physical contact therebetween. Thus, the isolated current sensor 100 measures a converted DC current without interrupting the circuit, thereby being transparent to the power source 106 and the load 104. The current sensor 100 is coupled to the controller 24 and supplies a signal corresponding to the measured amplitude of the direct current thereto. The controller 24 may process the current signal by using any type of a processing function that correlates the AC output of the power source 106 with the converted DC current. The measured DC current reflective of the output AC current may be used in a variety of ways, including algorithm control, dosage error checks, and other output verification.

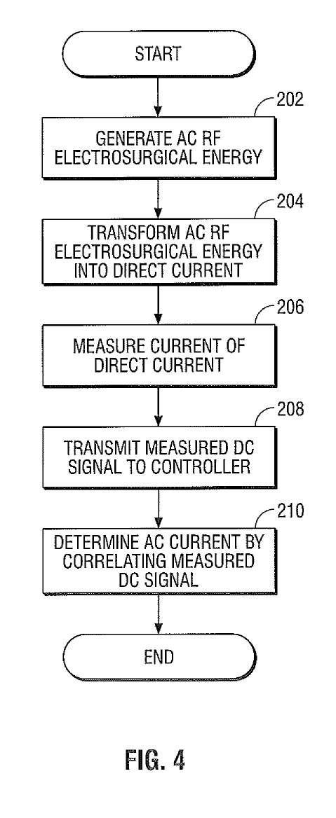

FIG. 4 shows a flow chart of a method 200 according to the present disclosure. The method includes a step 202 during which the power source 106, e.g., the output stage 28, outputs AC RF therapeutic electrosurgical energy to the load 104, e.g., patient tissue. In step 204, as the energy is transmitted to the load 104, the bridge rectifier 102 transforms a portion of the AC current into DC, which is then transmitted to the isolated current sensor 100. In step 206, the isolated current sensor 100 measures the variations in the DC current transmitted through the coupling member 101. In step 208, the current sensor 100 transmits the DC current signal to the controller 24, which in step 210, then correlates the signal to the AC current output to determine actual AC current output.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical generator comprising:
an output stage configured to generate at least one radio frequency electrosurgical waveform;
a bridge rectifier coupled to the output stage and configured to pass-through the at least one radio frequency electrosurgical waveform and to transform at least a portion of the at least one radio frequency electrosurgical waveform into direct current; and
an isolated current sensor configured to measure amplitude of the direct current.

2. The electrosurgical generator according to claim 1, wherein the isolated current sensor is a Hall Effect sensor.

3. The electrosurgical generator according to claim 1, wherein the bridge rectifier includes:
first and second output terminals coupled between the output stage and a load.

4. The electrosurgical generator according to claim 1, wherein the bridge rectifier includes:
first and second input terminals interconnected by at least one coupling member.

5. The electrosurgical generator according to claim 4, wherein the isolated current sensor is a Hall Effect sensor disposed in proximity to the at least one coupling member.

6. The electrosurgical generator according to claim 1, further comprising a controller coupled to the isolated current sensor, the controller configured to determine radio frequency current of the at least one radio frequency electrosurgical waveform based on the direct current measured by the isolated current sensor.

7. An electrosurgical system comprising:
at least one pair of electrodes coupled to a load;
an output stage coupled to the at least one pair of electrodes, the output stage configured to generate at least one radio frequency electrosurgical waveform;
a bridge rectifier coupled to the output stage and configured to pass-through the at least one radio frequency electrosurgical waveform and to transform at least a portion of the at least one radio frequency electrosurgical waveform into direct current; and
an isolated current sensor configured to measure amplitude of the direct current.

8. The electrosurgical system according to claim 7, wherein the isolated current sensor is a Hall Effect sensor.

9. The electrosurgical system according to claim 7, wherein the bridge rectifier includes:
first and second output terminals coupled between the output stage and a load.

10. The electrosurgical system according to claim 7, wherein the bridge rectifier includes:
first and second input terminals interconnected by at least one coupling member.

11. The electrosurgical system according to claim 10, wherein the isolated current sensor is a Hall Effect sensor disposed in proximity to the at least one coupling member.

12. The electrosurgical system according to claim 7, further comprising a controller coupled to the isolated current sensor, the controller configured to determine radio frequency current of the at least one radio frequency electrosurgical waveform based on the direct current measured by the isolated current sensor.

13. A method for electrosurgery, comprising:
supplying at least one radio frequency electrosurgical waveform from an output stage to at least one pair of electrodes;
passing the at least one radio frequency electrosurgical waveform to the at least one pair of electrodes through a bridge rectifier;
transforming at least a portion of the at least one radio frequency electrosurgical waveform into direct current; and
measuring current of the direct current.

14. The method according to claim 13, wherein the bridge rectifier includes:
first and second output terminals coupled between the output stage and a load.

15. The method according to claim 13, wherein the bridge rectifier includes:
first and second input terminals interconnected by at least one coupling member.

16. The method according to claim 15, wherein the measuring includes detecting the direct current through an isolated current sensor.

17. The method according to claim 16, wherein the isolated current sensor is a Hall Effect sensor disposed in proximity to the at least one coupling member.

18. The method according to claim 13, further comprising:
transmitting a signal corresponding to measured direct current to a controller.

19. The method according to claim 18, further comprising:
determining radio frequency current of the at least one radio frequency electrosurgical waveform based on the signal corresponding to measured direct current.
